(19) United States
(12) Patent Application Publication
(10) Pub. No.: US 2016/0339139 A1
(43) Pub. Date: Nov. 24, 2016

(54) Layered Structure Having Sequestered Oxygen Catalyst

(71) Applicant: AVENT, INC., Alpharetta, GA (US)

(72) Inventor: Shruti Aryal, Sao Paulo (BR)

(73) Assignee: AVENT, INC, Alpharetta, GA (US)

(21) Appl. No.: 15/113,877

(22) PCT Filed: Jan. 30, 2015
(86) PCT No.: PCT/US2015/013661

§ 371 Date: Jul. 25, 2016

Related U.S. Application Data
(60) Provisional application No. 61/934,149, filed on Jan. 31, 2014.

(51) Int. Cl. A61L 15/38; B01J 20/26; A61L 15/60; B01J 35/00; B01J 31/06; A61L 15/22; B01J 31/00; B01J 20/24

(52) U.S. Cl. CPC A61L 15/38; B01J 31/003; B01J 20/261; B01J 20/24; B01J 35/0006; B01J 31/06; A61L 15/225; A61L 15/60; A61L 2300/62

(57) ABSTRACT

An oxygen catalyst-containing structure comprising a first layer encapsulated by a second layer is provided, where the first layer includes an oxygen catalyst and the second layer is free of an oxygen catalyst. A method of making an oxygen catalyst-containing structure comprising a first layer and a second layer is also provided where the first layer includes an oxygen catalyst, the second layer is free of an oxygen catalyst, and the first layer is encapsulated by the second layer. The method includes impregnating a first solution containing a first superabsorbent polymer with an oxygen catalyst; allowing the first solution to gel to form the first layer; coating the first layer with a second solution containing a second superabsorbent polymer; and allowing the second solution to gel to form the second layer.

### RELATED APPLICATIONS

The present application claims priority to U.S. Provisional Application Ser. No. 61/934,149, filed on Jan. 31, 2014, which is incorporated herein in its entirety by reference thereto.

### BACKGROUND OF THE INVENTION

The present disclosure relates to a coating that contains oxygen that can be applied to various substrates.

Lack of oxygen (i.e., hypoxia) is commonly experienced by people in their extremities as they get older due to poor blood circulation as well as by people affected by conditions such as diabetes. Studies have also shown below normal, low oxygen tension in the skin of elderly people. This often leads to poor skin health and an excessive presence of visible conditions such as wrinkles, dryness, and lower skin elasticity. Over the years, cosmetic manufacturers have introduced skin formulations with a large variety of ingredients such as emollients, exfoliators, moisturizers, etc., in an attempt to retard these age related effects and improve and maintain skin health.

In addition to alleviating symptoms related to the normal decrease in oxygen delivery to the skin, oxygen applied to wounds as, for example, a dressing containing oxygen, can speed healing. The delivery of oxygen to the skin and wounds for common use is a technological challenge, since oxygen is quite reactive and unstable. As such, it has been difficult to provide high concentrations of oxygen for at home use because of this instability. Oxygen has, however, been provided in the form of a peroxide and a peroxide decomposition catalyst per U.S. Patent Application Publication No. 2006/0121101 to Ladizinsk . This publication provides such a treatment for intact skin through the use of a dressing that is applied to an area of the skin. The dressing generally has a rupturable reservoir containing an aqueous hydrogen peroxide composition and a hydrogel matrix layer having a peroxide decomposition catalyst. Unfortunately, the catalytic decomposition of hydrogen peroxide to oxygen is quite rapid and so the dressing includes a layer that is impermeable to oxygen on the outside so that the oxygen is held against the skin for the maximum time possible. While this dressing is useful for small areas of the skin, it is unworkable for large areas or irregularly shaped areas of skin.

Alternatively, U.S. Pat. No. 5,736,582 to Devillez proposes the use of hydrogen peroxide in the place of benzoyl peroxide in skin treatment compositions that also contain solvents for hydrogen peroxide. This allows the hydrogen peroxide to stay below a level that will damage the skin and to stay in solution in greater concentrations. A solvent such as dimethyl isosorbide along with water is taught as being effective in its skin treatment composition. No peroxide decomposition catalyst is present. Unfortunately, no data on oxygen concentration or generation are given, nor is the time required for oxygen liberation. While this method appears to be an advance over non-oxygen containing compositions, the lack of data makes it difficult to make objective judgments on the overall effectiveness of this approach. Given the concentrations of peroxide, however, it is doubtful that significant volumes of oxygen were generated.

U.S. Pat. No. 7,160,553 to Gibbins, et al. proposes a matrix made from a polymer network and a non-gellable polysaccharide having oxygen for the treatment of compromised tissue. A closed cell foam is used to contain the dissolved oxygen and can also deliver other active agents.

U.S. Pat. No. 5,792,090 to Ladin proposes a wound dressing having an oxygen permeable layer in contact with the skin with an oxygen solution supply reservoir proximate the oxygen permeable layer. The reservoir is adapted to receive an aqueous liquid capable of supplying oxygen through chemical reaction. Preferably, the aqueous liquid contains hydrogen peroxide and the reservoir contains an immobilized solid hydrogen peroxide decomposition catalyst such as manganese dioxide. The catalyst in the dressing generates oxygen upon the addition of hydrogen peroxide.

Despite the development of the aforementioned dressings, matrices, and compositions, a need currently exists for an easy-to-use technology that can impart the ability to deliver oxygen to the skin attached to various types of substrates such as plastics, foams, non-wovens, and paper based products. It would also be desirable to have this technology in a form such that it is amenable for continuous manufacturing processes rather than batch type processes. A need also exists for a stable oxygen delivery product that can deliver oxygen on demand but that also separates the oxygen catalyst from the outer surface of the product, as oxygen catalysts can cause skin irritation.

### SUMMARY OF THE INVENTION

In accordance with one embodiment of the present invention, an oxygen catalyst-containing structure is disclosed having a first layer encapsulated by a second layer, where the first layer includes an oxygen catalyst and the second layer is free of an oxygen catalyst. In one embodiment, the first layer can include a first superabsorbent polymer such that the first layer contains between 80 wt. % and 99 wt. % of the first superabsorbent polymer and between 1 wt. % and 20 wt. % of the oxygen catalyst on a water-free basis. In another embodiment, the first layer can contain between 80 wt. % and 90 wt. % of the first superabsorbent polymer and between 1 wt. % and 10 wt. % of the oxygen catalyst on a water-free basis. The first superabsorbent polymer can include polyacrylamide, polyacrylate, agar, or a combination thereof, and, in some embodiments, the first superabsorbent polymer can further include a non-gellable polysaccharide. Meanwhile, the oxygen catalyst can be sodium carbonate, manganese dioxide, or catalase.

In yet another embodiment, the second layer of the structure can include a second superabsorbent polymer. The second superabsorbent polymer can include polyacrylamide, polyacrylate, agar, or a combination thereof, and, in some embodiments, the second superabsorbent polymer can further include a non-gellable polysaccharide. In still other embodiments, the second layer can be perforated.

In yet another embodiment, the structure of the present invention can include one or more additional layers, and the one or more additional layers can be a bandage, gauze, film, or mesh.

In an additional embodiment, at least about 95% of the oxygen catalyst can be sequestered within the structure. In one more embodiment, at least about 99% of the oxygen catalyst is sequestered within the structure.

In accordance with another embodiment of the present invention, a method of making an oxygen catalyst-containing structure that includes a first layer and a second layer is disclosed. The first layer contains an oxygen catalyst and the second layer is free of an oxygen catalyst, and the first layer is encapsulated by the second layer. The method includes impregnating a first solution containing a first superabsorbent polymer with an oxygen catalyst; allowing the first solution to gel to form the first layer; coating the first layer with a second solution containing a second superabsorbent polymer; and allowing the second solution to gel to form the second layer.

In one embodiment, the first layer can include a first superabsorbent polymer, wherein the first layer comprises between 80 wt. % and 99 wt. % of the first superabsorbent polymer and between 1 wt. % and 20 wt. % of the oxygen catalyst on a water-free basis. Further, the first superabsorbent polymer can include polyacrylamide, polyacrylate, agar, or a combination thereof, while the second layer can include a second superabsorbent polymer, which can include polyacrylamide, polyacrylate, agar, or a combination thereof.

In yet another embodiment, the oxygen catalyst can include sodium carbonate, manganese dioxide, or catalase.

Other features and aspects of the present invention are set forth in greater detail below.

### BRIEF DESCRIPTION OF THE FIGURES

A full and enabling disclosure of the present invention, including the best mode thereof to one skilled in the art, is set forth more particularly in the remainder of the specification, including reference to the accompany figures, in which:

FIG. 1 is a cross-sectional view of a schematic of one embodiment of a layered structure contemplated by the present invention;

FIG. 2 is a top view of a photograph of one embodiment of a layered structure contemplated by the present invention; and

FIG. 3 is a top view of a photograph of one embodiment of a layered structure contemplated by the present invention after being contacted with a peroxide-containing lotion.

Repeat use of reference characters in the present specification and drawings is intended to represent the same or analogous features or elements of the present invention.

### DETAILED DESCRIPTION

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

Generally speaking, the present invention is directed to an oxygen catalyst containing structure that includes an oxygen catalyst containing layer (e.g., a first layer) overcoated with an additional layer that is free of an oxygen catalyst (e.g., a second layer). In other words, the first layer is encapsulated by the second layer. In this manner, the oxygen containing cells are non-uniformly distributed in the structure such that the catalyst is sequestered in a layer that does not come in contact with the skin when the structure is used as a wound dressing.

In the process of making the structure with an oxygen catalyst containing layer, a superabsorbent polymer can be synthesized and an oxygen catalyst (sodium carbonate, manganese dioxide, catalase, etc.) can be added during polymerization, after which the oxygen catalyst containing layer can be allowed to gel. Next, a second layer without an oxygen catalyst is coated in solution form onto the first, oxygen catalyst containing layer, or, alternatively, the first layer can be dipped into the solution, where the second layer is formed around the first layer. As a result, the oxygen catalyst is sequestered inside an interior of the structure such that the oxygen catalyst can be prevented from coming into direct contact with skin when the structure is applied as, for instance, a wound dressing.

Various embodiments of the present invention will now be described in further detail.

In the process of making the layered structure disclosed herein, a superabsorbent material (e.g., a superabsorbent polymer as discussed in more detail below) is synthesized using established procedures and processes. As is known in the art, a small amount of polymerization catalyst like bis-acrylamide can be used to polymerize the acrylamide or acrylate. To make the first layer, during the polymerization process, an oxygen catalyst is added, although it is to be understood that an oxygen catalyst is not utilized during the polymerization process to make the second layer. The oxygen catalyst can include sodium carbonate, manganese dioxide, catalase, etc. The oxygen catalyst is not believed to take part in the polymerization reaction that produces the superabsorbent polymer. The superabsorbent polymer and oxygen catalyst polymer mixture thus produced is then dried until it is water-free. The resulting first layer can include between 80 wt. % and 99 wt. % of a superabsorbent polymer and between 1 wt. % and 20 wt. % of an oxygen catalyst on a water-free basis, such as between 85 wt. % and 97.5 wt. % of the superabsorbent polymer and between 2.5 wt. % and 15 wt. % of the oxygen catalyst on a water-free basis. In one particular embodiment, the first layer can include between 80 wt. % and 90 wt. % of the superabsorbent polymer and between 1 wt. % and 10 wt. % of the oxygen catalyst on a water-free basis. By “water-free” is meant the condition of the mixture after dehydrating or drying down to a moisture loss of between 60% and 80%. Meanwhile, the second layer excludes the oxygen catalyst such that the second layer can include between 85 wt. % and 100 wt. % of a superabsorbent polymer on a water-free basis, such as from about 90 wt. % and 99.9 wt. %, such as from about 95 wt. % to about 99 wt. % of a superabsorbent polymer on a water-free basis.

Typically, a superabsorbent polymer is capable of absorbing at least about 10 times its weight in a 0.9 weight percent aqueous sodium chloride solution, and particularly is capable of absorbing more than about 20 times its weight in 0.9 weight percent aqueous sodium chloride solution. Superabsorbent polymers suitable for treatment or modification in accordance with the present invention are available from various commercial vendors, such as Dow Chemical Company located in Midland, Mich., USA, and Stockhausen Inc., Greensboro, N.C., USA. Other superabsorbent polymers suitable for treatment or modification in accordance with the present invention are described in U.S. Pat. No. 5,601,542 to Melius, et al.; U.S. Patent Application Publication No. 2001/0049514 to Dodge, et al.; and, U.S. patent application Ser. No. 09/475,830 to Dodge, et al.; each of which is hereby incorporated by reference in a manner consistent herewith.

Suitable superabsorbent materials useful in the present disclosure may be selected from natural, synthetic, and modified natural polymers and materials. The superabsorbent materials may be inorganic materials, such as silica gels, or organic compounds, including natural materials such as agar, agarose, pectin, a non-gellable polysaccharide (guar gum, lucerne, fenugreek, honey locust bean gum, white clover bean gum, carob locust bean gum, etc.), collagen, gelatin, chondroitin, calmodulin, cellulose, dextran, alginate, and the like.

The superabsorbent materials may also be synthetic materials, such as synthetic hydrogel matrix polymers. Such hydrogel matrix polymers include, for example, alkali metal salts of polyacrylic acids; polyacrylamides; polyvinyl alcohol; ethylene maleic anhydride copolymers; polyvinyl ethers;

hydroxypropylcellulose; polyvinyl morpholinone; polymers and copolymers of vinyl sulfonic acid, polyacrylates, polyacrylamides, polyvinyl pyridine; polyamines; and, combinations thereof. Other suitable polymers include hydrolyzed acrylonitrile grafted starch, acrylic acid grafted starch, and isobutylene maleic anhydride copolymers and combinations thereof. The superabsorbent materials of the present disclosure may be in any form suitable for use in absorbent structures, including, particles, fibers, flakes, spheres, and the like. The hydrogel matrix polymers may be suitably lightly crosslinked to render the material substantially water-insoluble. Crosslinking may, for example, be by irradiation or by covalent, ionic, Van der Waals, or hydrogen bonding. One suitable cross-linking agent is N,N′-methylene-bisacrylamide, however other appropriate cross-linking agents such as bisacrylylycystamine and diallyltartar diamide may also be used. If N,N′-methylene-bisacrylamide or any other suitable cross-linking agent is used, it can be a component of the first layer and/or the second layer of the structure of the present invention in an amount ranging from about 0.005 wt. % to about 0.5 wt. %, such as from about 0.01 wt. % to about 0.25 wt. %, such as from about 0.025 wt. % to about 0.15 wt. % based on a water-free basis. Ammonium persulfate and tetramethylethylenediamine (TEMED) may also be added to the matrix. The ammonium persulfate can be a component of the first layer and/or the second layer of the structure of the present invention in an amount ranging from about 0.005 wt. % to about 0.5 wt. %, such as from about 0.01 wt. % to about 0.25 wt. %, such as from about 0.025 wt. % to about 0.1 wt. % based on a water-free basis. Additionally, TEMED can be a component of the first layer and/or the second layer of the structure of the present invention in an amount ranging from about 0.001 wt. % to about 0.5 wt. %, such as from about 0.01 wt. % to about 0.25 wt. %, such as from about 0.025 wt. % to about 0.15 wt. % based on a water-free basis.

Any layer of the structure of the present invention may also contain other excipients like humectants and/or plasticizers such as glycerin, propylene glycol, polyethylene glycol (PEG), etc.

The structure may be coated onto the desired substrate by extrusion, roll to roll coating, spin coating, or any other suitable processes. Alternatively, the structure may remain free flowing and applied to a wound or substrate as a liquid.

Regardless of whether the structure is applied to a substrate or remains free flowing, the structure may be exposed to hydrogen peroxide by, for example, dipping the structure into a hydrogen peroxide solution or lotion. Alternatively, a substrate on which the structure is coated may be sprayed with hydrogen peroxide. When the structure is exposed to hydrogen peroxide, the catalyst containing layer foams, indicating that oxygen is being liberated from the layered structure despite the oxygen catalyst being contained in only the first layer of the structure, which is encapsulated by the second layer.

Unlike oxygen containing structures in which the oxygen containing cells are uniformly distributed, the structure of the present invention provides separation between a first layer of closed cells containing gaseous oxygen within a multilayer structure where the second layer adjacent the first layer does not have oxygen containing cells (e.g., is free of an oxygen catalyst). Such separation is important in that it prevents the oxygen catalyst from contacting the skin and it allows additional functionalities to be included or accentuated into the structure. For example, a uniform, distributed through-out, arrangement (as a coating for example) is believed to be limited in wicking watery exudate away from a wound because of blocking or impeding of flow by other oxygen containing cells. On the other hand, because the structure of the present invention includes a second layer containing a superabsorbent material in which no oxygen catalyst is present, where the second layer is the skin-contacting layer of the structure, the second layer can wick exudate away from a wound on the skin. This phenomenon is known in the art of producing personal care products containing superabsorbent materials as “gel blocking”. In the non-uniform, layered structure as provided herein, the parts of the structure not containing oxygen cells are able to more easily conduct water between the oxygen cell clusters, allowing for enhanced wicking.

In one particular embodiment, the layered structure of the present invention includes a first layer that includes an agar-based hydrogel matrix impregnated with an oxygen catalyst and a second layer that is a polyacrylamide-based hydrogel matrix that is free of an oxygen catalyst, where such an arrangement creates a non-uniform structure. However, it is to be understood that any suitable superabsorbent polymer or combination thereof can be used in the first and second layers. Once second layer gels around first layer that contains the oxygen catalyst, the entire structure can be dried down (dehydrated), and the oxygen catalyst can be successfully sequestered. It is also to be understood that additional layers containing alternate functionality may be added over the two layer structure described above to produce a multilayered structure. For instance, a bandage, gauze, film, or mesh layer may be added, for example, for ease of handling or to maintain the structure in a desired location. In this manner a bandage-like structure or dressing for application to a wound may be produced.

In an additional embodiment, the second layer may be perforated. The perforations in the second layer allow for wound exudate to flow through the resulting structure or dressing, making it more suitable for wounds that have a larger amount of drainage. The perforations could also improve oxygen permeability through the structure and to the wound.

The general procedure for impregnating, encapsulating, or sequestering a catalyst into a superabsorbent material to form the first layer of the structure of the present invention includes preparing a monomer mix of the superabsorbent polymer, which can also include other optional ingredients such as a plasticizer (e.g., glycerol), a non-gellable polysaccharide (e.g., guar gum), etc. can be combined in water to form a first solution. Then activators such as tetramethylethylenediamine (TEMED) and ammonium persulfate can be added to the first solution while the first solution is mixing. Soon after adding any activators, a catalyst (e.g., catalase) is added into the first solution. The first solution can be mixed for about 3 minutes to about 5 minutes to form a homogenous solution. Immediately thereafter, the first solution can be poured into an appropriate mold or container and allowed to gel to form a first hydrogel matrix. Then, a second solution from which the second layer of the structure is formed can be made in a similar manner as the solution for the first layer, but without the addition of the catalyst. After the second solution for the second layer is formed, the second solution can be poured over the already-formed first layer and allowed to gel to form a coating of a second hydrogel matrix that surrounds the first hydrogel matrix, resulting in a structure in which the first layer (e.g., first hydrogel matrix) is surrounded by the second layer (e.g., second hydrogel matrix), which prevents the catalyst in the first layer from contacting the skin when the structure is applied as a wound dressing. Alternatively, the first layer can be dipped into the second solution to form a coating of the second layer around the first layer.

In one particular embodiment when the superabsorbent material is agar, the procedure for impregnating, encapsulating, or sequestering a catalyst into the agar is as follows. First, an agar solution is prepared, where the agar is dissolved in water at a concentration between about 1 wt. % and about 2 wt. %. A low melting agar can be used so that the agar does not solidify too quickly at a temperature near body temperature (e.g., about 37° C.). Then, the agar is melted by boiling the solution or by autoclaving the solution. If the solution is autoclaved, the agar should be hydrated in water for at least about 2 hours prior to autoclaving the solution. After boiling or autoclaving, the agar solution is cooed down in a water bath until the agar solution reaches a temperature between about 40° C. and about 52° C. Once the agar solution is cooled to the desired temperature, the desired amount of catalyst solution can be added to the agar solution. The resulting solution (first solution) is mixed, such as by vortexing, and then the solution is poured into a mold or container such that it can solidify into a first layer (e.g., first hydrogel matrix). Thereafter, a second solution can be formed that does not include a catalyst. After the second solution for the second layer is formed, the second solution can be poured over the already-formed first layer and allowed to gel to form a coating of a second hydrogel matrix that surrounds the first hydrogel matrix, resulting in a structure in which the first layer (e.g., first hydrogel matrix) is surrounded by the second layer (e.g., second hydrogel matrix), which prevents the catalyst in the first layer from contacting the skin when the structure is applied as a wound dressing. Alternatively, the first layer can be dipped into the second solution to form a coating of the second layer around the first layer.

After the structure containing a first layer encapsulated within a second layer is formed, the structure can be dried in an oven at a temperature of up to about 55° C. for a time period of up to about 17 hours without loss of catalytic activity contained within the first layer. After the desired level of drying is achieved, the structure is ready for use in conjunction with a peroxide reservoir to generate oxygen on demand, where the structure is capable of decomposing the peroxide to generate the oxygen despite the sequestration of the catalyst in the encapsulated first layer of the structure. However, it is also to be understood that it is not required that the structure be dried, and, instead, a hydrated form of the structure can be utilized in conjunction with a peroxide solution to generate oxygen on demand, where the structure is capable of decomposing the peroxide to generate the oxygen.

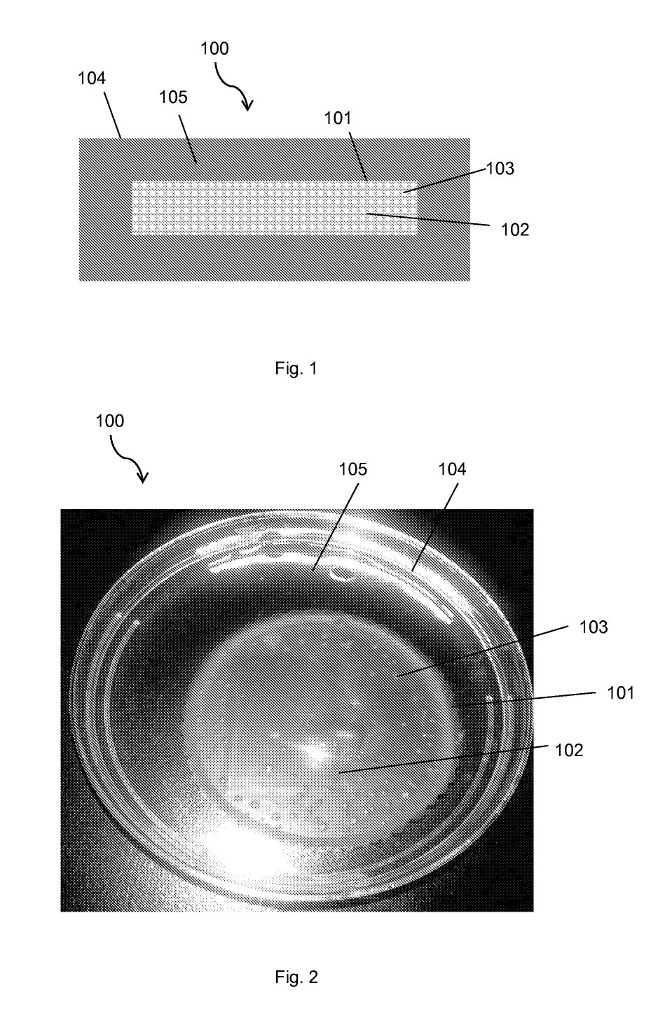

Referring now to FIGS. 1-3, the layered structure of the present invention is shown before and after use. First, FIG. 1 shows a cross-sectional view of a layered structure 100 having a first layer 101 that includes a superabsorbent polymer 102 and oxygen containing cells 103 formed by the inclusion of an oxygen catalyst in the first layer 101. The first layer 101 is surrounded or encapsulated by a second layer 104 that includes a superabsorbent polymer 105.

Meanwhile, FIG. 2 is a top view of a photograph of the layered structure 100 showing the oxygen containing cells 103 distributed throughout a superabsorbent polymer 102 to form the first layer 101, the second layer 104 includes a superabsorbent polymer 105.

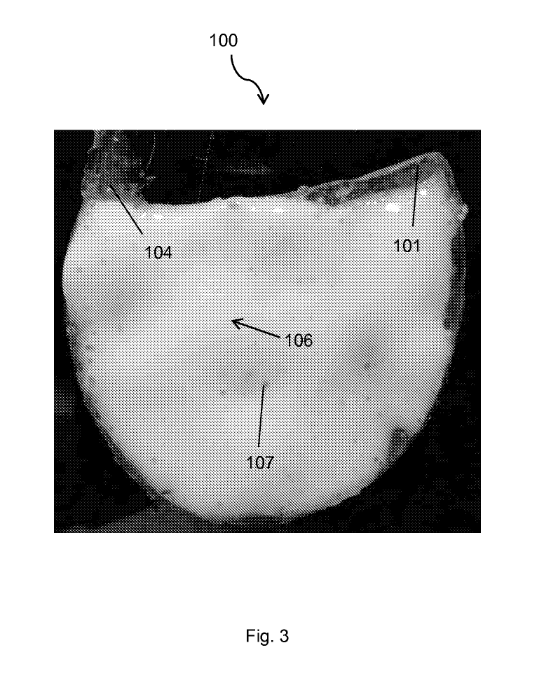

Further, FIG. 3 demonstrates the generation of oxygen on demand when a peroxide-containing lotion 106 is placed in contact with the structure 100 of FIG. 2, where foaming 107 occurs, indicating that oxygen is being liberated when the lotion 106 contacts the first layer 101, which includes the oxygen catalyst.

The present invention may be better understood with reference to the following examples.

### EXAMPLE 1

The ability to form a two-layer structure including a first layer containing polyacrylamide and catalase surrounded by a second layer containing polyacrylamide without catalase is demonstrated.

First, acrylamide, glycerol, and guar gum were combined in water to form a first solution. Then tetramethylethylenediamine (TEMED) and ammonium persulfate were added to the first solution while the first solution was mixing. Next, catalase was added into the same solution. The solution was then mixed for about 3 minutes to about 5 minutes to form a homogenous solution. Immediately thereafter, the solution was poured into a petri dish and allowed to gel to form a first layer. Then, a second solution was made in the same manner as the first solution used to form the first layer, but without the addition of the catalase. After the solution for the second layer was formed, it was poured over the gelled first layer and allowed to gel, resulting in a structure in which the first layer is surrounded or encapsulated by the second layer. The structure was then dried at 55° C. for 17 hours to reach a moisture loss of about 60% to about 80%, after which the structure was stored until it was ready for use. The structure contained 15660 U of catalase.

### EXAMPLE 2

The ability to form a two-layer structure including a first layer containing agar and catalase surrounded by a second layer containing agar without catalase is demonstrated.

First, agar (commercially available from Fisher Scientific) was impregnated with catalase by forming a 1% to 2% agar solution in water, melting the agar by boiling, and cooling the agar down in water bath to a temperature of about 48° C. Then, a catalase solution including catalase from BioCat of Troy, Va. was added to the agar solution to form a first solution having a final catalase activity level of 1500 U/g. The solution was mixed thoroughly and poured into a petri dish to solidify to form a first layer. Then, a second solution was made in the same manner as the first solution used to form the first layer, but without the addition of the catalase. After the solution for the second layer was formed, it was poured over the gelled first layer and allowed to gel, resulting in a structure in which the first layer is surrounded or encapsulated by the second layer. The structure was then dried at 55° C. for 17 hours to reach a moisture loss of about 60% to about 80%, after which the structure was stored until it was ready for use.

### EXAMPLE 3

The ability of the structure of Example 1 to successfully sequester catalase in the first layer of the structure is demonstrated, which corresponds with the enhanced stability of the structure of Example 1 as well as the ability of the first layer to prevent direct contact of the catalase with skin.

After drying, the structure of Example 1 was soaked in deionized water for a time period of 24 hours. A 1 milliliter aliquot of the resulting soaking liquid was then removed and allowed to react with 1 milliliter of 0.9% hydrogen peroxide for a time period of 5 minutes (Test Sample) to test for hydrogen peroxide decomposition to see if there is any catalytic activity, where catalytic activity indicates loss of catalyst from the structure. Further, a 1 milliliter aliquot containing 5.2 U of catalase was used as a control and was allowed to react with 1 milliliter of 0.9% hydrogen peroxide for a time period of 5 minutes (Control Sample). Then, the decomposition of the peroxide was measured for each sample. The results showed that 74% of the hydrogen peroxide decomposed during the reaction for the Control Sample, while only 60% of the hydrogen peroxide decomposed during the reaction for the Test Sample. Thus, this indicates that less than 5.2 U/mL of catalase was present in the resulting soaking liquid since the Test Sample exhibited lower hydrogen peroxide decomposition than the Control Sample that included 5.2 U/mL of catalase. Considering that 15660 U of catalase was included in the structure of Example 1, this corresponds with a 99.97% sequestration of catalase within the first layer of the two-layered structure, where (15660 U−5.2 U)/(15660−U)*100=99.97%.

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. An oxygen catalyst-containing structure comprising a first layer encapsulated by a second layer, wherein the first layer includes an oxygen catalyst and the second layer is free of an oxygen catalyst.

2. The structure of claim 1, wherein the first layer comprises a first superabsorbent polymer, wherein the first layer comprises between 80 wt. % and 99 wt. % of the first superabsorbent polymer and between 1 wt. % and 20 wt. % of the oxygen catalyst on a water-free basis.

3. The structure of claim 1, wherein the first layer comprises between 80 wt. % and 90 wt. % of the first superabsorbent polymer and between 1 wt. % and 10 wt. % of the oxygen catalyst on a water-free basis.

4. The structure of claim 2, wherein the first superabsorbent polymer comprises polyacrylamide, polyacrylate, agar, or a combination thereof.

5. The structure of claim 4, wherein the first superabsorbent polymer further comprises a non-gellable polysaccharide.

6. The structure of claim 1, wherein the oxygen catalyst comprises sodium carbonate, manganese dioxide, or catalase.

7. The structure of claim 1, wherein the second layer comprises a second superabsorbent polymer.

8. The structure of claim 7, wherein the second superabsorbent polymer comprises polyacrylamide, polyacrylate, agar, or a combination thereof.

9. The structure of claim 8, wherein the second superabsorbent polymer further comprises a non-gellable polysaccharide.

10. The structure of claim 1, wherein the second layer is perforated.

11. The structure of claim 1, further comprising one or more additional layers.

12. The structure of claim 11, wherein the one or more additional layers is a bandage, gauze, film, or mesh.

13. The structure of claim 1, wherein at least about 95% of the oxygen catalyst is sequestered within the structure.

14. The structure of claim 1, wherein at least about 99% of the oxygen catalyst is sequestered within the structure.

15. A method of making an oxygen catalyst-containing structure comprising a first layer and a second layer, wherein the first layer includes an oxygen catalyst and the second layer is free of an oxygen catalyst, further wherein the first layer is encapsulated by the second layer, the method comprising:
impregnating a first solution containing a first superabsorbent polymer with an oxygen catalyst;
allowing the first solution to gel to form the first layer;
coating the first layer with a second solution containing a second superabsorbent polymer; and
allowing the second solution to gel to form the second layer.

16. The method of claim 15, wherein the first layer comprises a first superabsorbent polymer, wherein the first layer comprises between 80 wt. % and 99 wt. % of the first superabsorbent polymer and between 1 wt. % and 20 wt. % of the oxygen catalyst on a water-free basis.

17. The method of claim 16, wherein the first superabsorbent polymer comprises polyacrylamide, polyacrylate, agar, or a combination thereof.

18. The method of any one of claim 15, wherein the second layer comprises a second superabsorbent polymer.

19. The method of claim 18, wherein the second superabsorbent polymer comprises polyacrylamide, polyacrylate, agar, or a combination thereof

20. The method of claim 15, wherein the oxygen catalyst comprises sodium carbonate, manganese dioxide, or catalase.
